(19) United States
(12) Patent Application Publication
(10) Pub. No.: US 2010/0098671 A1
(43) Pub. Date: Apr. 22, 2010

(54) COMPOSITION FOR PREVENTION OR TREATMENT OF BONE MARROW DAMAGE

(75) Inventors: Youngsook Son, Seoul (KR); Hyunsook Hong, Seoul (KR); Jungsun Lee, Seoul (KR)

Correspondence Address: ROTHWELL, FIGG, ERNST & MANBECK, P.C., 1425 K STREET, N.W., SUITE 800, WASHINGTON, DC, 20005, US

(73) Assignees: MODERN CELL & TISSUE TECHNOLOGIES INC., Seoul (KR); KOREA INSTITUTE OF RADIOLOGICAL & MEDICAL SCIENCES, Seoul (KR)

(21) Appl. No.: 12/524,127

(22) PCT Filed: Jun. 7, 2007
(86) PCT No.: PCT/KR07/02762

§ 371 Date: Oct. 20, 2009

(30) Foreign Application Priority Data
Jan. 22, 2007 (KR) 10-2007-0006409

(51) Int. Cl. A61K 38/08; C07K 7/06; A61K 35/12; A61P 35/00

(52) U.S. Cl. 424/93.7; 530/327; 514/15

(57) ABSTRACT

Disclosed herein are a composition for prevention or treatment of bone marrow damage comprising Substance-P as an active ingredient; a use of Substance-P for the preparation of a medicament for prevention or treatment of bone marrow damage; a method for prevention or treatment of bone marrow damage comprising administering a therapeutically effective amount of Substance-P to a mammal; an anticancer supplement comprising Substance-P as an active ingredient; a use of Substance-P for the preparation of an anticancer supplement; and a method for prevention or treatment of cancer comprising administering a therapeutically effective amount of Substance-P as an anticancer supplement to a mammal.

Substance-P stimulates proliferation of mesenchymal stem cells (MSCs) within the bone marrow to thereby facilitate protection and regeneration of bone marrow cells and hematopoietic stem cells. Therefore, the composition of the present invention can be therapeutically used for treatment and/or prevention of bone marrow damage. Further, the composition of the present invention can be used as an anticancer supplement for anticancer therapy.

### TECHNICAL FIELD

The present invention relates to a composition for prevention or treatment of bone marrow damage comprising Substance-P as an active ingredient; a use of Substance-P for the preparation of a medicament for prevention or treatment of bone marrow damage; a method for prevention or treatment of bone marrow damage comprising administering a therapeutically effective amount of Substance-P to a mammal; an anticancer supplement comprising Substance-P as an active ingredient; a use of Substance-P for the preparation of an anticancer supplement; and a method for prevention or treatment of cancer comprising administering a therapeutically effective amount of Substance-P as an anticancer supplement to a mammal.

### BACKGROUND ART

Bone marrow damage, which is caused by various factors such as radiation exposure, administration of anticancer drugs, trauma, and the like, may result in the damage of hematopoietic stem cells (HSCs) as well as mesenchymal stem cells (MSCs) which are bone marrow stromal cells (BMSCs). MSCs are known to support the proliferation of HSCs. Further, it has been reported that it is possible to effectively facilitate ex vivo expansion of

HSCs when MSCs derived from human placenta, bone marrow or umbilical cord blood are used as a cell feeder layer (Zhang Y, et al., Exp Hematol 32:657-664, 2004; and Wang J F, et al., Haematologica 89:837-844, 2004). Therefore, MSCs can play an important role in spontaneous recovery of HSCs in damaged bone marrow or engraftment of transplanted HSCs.

Substance P (SP) is an 11-amino acid neuropeptide, which is expressed in sensory neurons, macrophages, eosinophils, corneal cells including endothelium, epithelium and keratocytes, and granulation tissues. Several reports have suggested implication of Substance-P in neuro-immune communication on hematopoietic regulation. Substance-P nerve fibers are distributed in the bone marrow stroma, and Substance-P binds to the neurokinin-1 receptor (NK-1) to thereby stimulate bone marrow stromal cells, resulting in production of stem cell factors and interleukin-1 which may be favorable for hematopoietic stimulation as feeders.

In a previous study as set forth by the present inventors, which addresses the role of

Substance-P in mobilization and repopulation of mesenchymal stem cells (MSCs), Korean Patent No. 10-593397 discloses a use of Substance-P for the manufacture of a medicament for wound-healing or facilitating wound-healing, and a use of Substance-P for the manufacture of a medicament for mobilization or proliferation of MSCs from the bone marrow, or facilitating the mobilization or proliferation of MSCs.

Based on results of the above-mentioned previous study, the present inventors made an attempt to investigate whether Substance-P is capable of exhibiting therapeutic effects on bone marrow damage through stimulation of the mesenchymal stem cell repopulation to thereby facilitate proliferation of bone marrow cells and hematopoietic stem cells, in bone marrow damage with accompanying cellular destruction of bone marrow cells and hematopoietic stem cells.

### DISCLOSURE OF THE INVENTION

### Technical Problem

Therefore, the present invention has been made in view of the above problems, and it is an object of the present invention to provide a composition for prevention or treatment of bone marrow damage comprising Substance-P as an active ingredient.

It is another object of the present invention to provide a use of Substance-P for the preparation of a medicament for prevention or treatment of bone marrow damage.

It is a further object of the present invention to provide a method for prevention or treatment of bone marrow damage comprising administering a therapeutically effective amount of Substance-P to a mammal.

It is another object of the present invention to provide an anticancer supplement comprising Substance-P as an active ingredient.

It is a still further object of the present invention to provide a use of Substance-P for the preparation of an anticancer supplement.

It is yet another object of the present invention to provide a method for prevention or treatment of cancer comprising administering a therapeutically effective amount of Substance-P as an anticancer supplement to a mammal.

### Technical Solution

In accordance with an aspect of the present invention, the above and other objects can be accomplished by the provision of a composition for prevention or treatment of bone marrow damage comprising Substance-P as an active ingredient; a use of Substance-P for the preparation of a medicament for prevention or treatment of bone marrow damage; and a method for prevention or treatment of bone marrow damage comprising administering a therapeutically effective amount of Substance-P to a mammal.

In accordance with another aspect of the present invention, there is provided an anticancer supplement comprising Substance-P as an active ingredient; a use of Substance-P for the preparation of an anticancer supplement; and a method for prevention or treatment of cancer comprising administering a therapeutically effective amount of Substance-P as an anticancer supplement to a mammal.

### ADVANTAGEOUS EFFECTS

The composition of the present invention can be therapeutically used for treatment and/or prevention of bone marrow damage. Further, the composition of the present invention can be used as an anticancer supplement for anticancer therapy.

### DESCRIPTION OF THE DRAWINGS

FIG. 1 is a graph comparing changes in the number of viable bone marrow cells between a group with administration of Substance-P prior to irradiation and a group without administration.

FIG. 2 is a graph comparing changes in the number of viable bone marrow cells between a group with administration of Substance-P after irradiation and a group without administration.

FIG. 3 is a graph comparing changes in the number of viable blood cells between a group with administration of Substance-P after irradiation and a group without administration.

FIG. 4 is a micrograph showing colony morphology after 2-week culture of bone marrow cells in HSC-CFU media, for a group with administration of Substance-P prior to irradiation and a group without administration.

FIG. 5 is a graph showing changes in colony counts after 2-week culture of bone marrow cells in HSC-CFU media, for a group with administration of Substance-P prior to irradiation and a group without administration.

FIG. 6 is a micrograph showing colony morphology after 2-week culture of bone marrow cells in HSC-CFU media, for a group with administration of Substance-P after irradiation and a group without administration.

FIG. 7 is a graph showing changes in colony counts after 2-week culture of bone marrow cells in HSC-CFU media, for a group with administration of Substance-P after irradiation and a group without administration.

### MODE FOR INVENTION

Hereinafter, the present invention will be described in more detail.

The present invention provides a composition for prevention or treatment of bone marrow damage comprising Substance-P as an active ingredient. In addition, the present invention provides a use of Substance-P for the preparation of a medicament for prevention or treatment of bone marrow damage.

In the present invention, bone marrow damage may be caused by various factors such as irradiation (radiotherapy), anticancer drug administration (chemotherapy), trauma, and the like. Irradiation may include radiation application for cancer radiotherapy, as well as accidental radiation exposure. Bone marrow damage caused due to other causal factors also falls within the scope of the present invention.

In order to demonstrate prophylactic and therapeutic effects of Substance-P on bone marrow damage in the present invention, bone marrow damage was induced by radiation in mice. Then, for comparison, observation was made of the number of bone marrow cells and blood cells of the Substance-P administered group and the non-administered group, and a colony count was also performed according to colony-forming unit (CFU) assay for each group. As a result, the Substance-P administered group was significantly higher in numbers of bone marrow cells and blood cells as well as in the colony count, as compared to the non-administered group, thus confirming that Substance-P stimulates mesenchymal stem cells to thereby activate the production of bone marrow cells and hematopoietic stem cells. Not only when Substance-P was administered to animals following induction of bone marrow damage, but also when animals were pretreated with Substance-P prior to induction of bone marrow damage, both exhibited significantly greater numbers of bone marrow cells and blood cells in conjunction with a significantly higher colony count, as compared to the non-administered group.

Therefore, it is possible to achieve treatment of bone marrow damage as well as effective prevention of bone marrow damage, when the composition of the present invention comprising Substance-P as an active ingredient is used therapeutically for bone marrow damage.

When the occurrence of bone marrow damage is inevitable due to radiotherapy or chemotherapy, for example, pretreatment of a subject with Substance-P prior to application of anticancer therapy can lead to significant reduction in the extent of bone marrow damage.

Further, administration of Substance-P in combination with conventional anticancer therapy can prevent decreases of blood cells due to the anticancer therapy, which then enables continuous chemotherapy regimens.

Consequently, prevention, treatment or alleviation of bone marrow damage resulting from conventional anticancer therapy can be accomplished via administration of the composition or medicament for prevention or treatment of bone marrow damage according to the present invention, concurrently with, before or after radiotherapy or chemotherapy.

Further, the composition of the present invention comprising Substance-P as an active ingredient can be used as an anticancer supplement. Therefore, the present invention provides an anticancer supplement comprising Substance-P as an active ingredient and a use of Substance-P for the preparation of an anticancer supplement.

The composition or medicament comprising Substance-P as an active ingredient or the anticancer supplement comprising Substance-P as an active ingredient may comprise one or more pharmaceutically acceptable carriers known in the art. After selection of the pharmaceutically acceptable carriers which are suitable for the administration of Substance-P to a subject, Substance-P may be suspended or dissolved in pharmaceutically acceptable carriers by a conventional method known in the art. Preferred is a water-soluble carrier.

As the incidence of bone marrow damage leads to a decreased number of mesenchymal stem cells in bone marrow, it is obvious that therapeutic effects on bone marrow damage will significantly increase by co-administration of mesenchymal stem cells capable of supporting production of hematopoietic stem cells in conjunction with Substance-P. The composition or medicament of present invention for prevention or treatment of bone marrow damage or the anticancer supplement of present invention may further comprise mesenchymal stem cells in addition to Substance-P.

As used herein, the term “MSC” or “mesenchymal stem cell” designates a cell which is capable of proliferating and differentiating into chondrogenic, osteogenic, adipogenic, stromogenic (marrow stromal), myogenic, and neural lineages. Mesenchymal stem cells may be derived from, e.g., bone marrow, umbilical cord blood, peripheral blood and other tissues.

Further, the present invention provides a method for prevention or treatment of bone marrow damage comprising administering a therapeutically effective amount of Substance-P to a mammal.

Further, the present invention provides a method for prevention or treatment of cancer comprising administering a therapeutically effective amount of Substance-P as an anticancer supplement to a mammal.

As used herein, the term “mammal” refers to any mammalian species that is in need of treatment, examination or experiment, preferably human.

As used herein, the term “therapeutically effective amount” refers to an amount of an active ingredient or pharmaceutical composition that will elicit the biological or medical response of a tissue system, animal or human that is being sought by a researcher, veterinarian, medical doctor or clinician, and encompasses an amount of the active ingredient or pharmaceutical composition which will relieve the symptoms of the disease or disorder being treated. As will be apparent to those skilled in the art, the therapeutically effective dose and administration times of the active ingredient in accordance with the present invention may vary depending upon desired therapeutic effects. Therefore, an optimal dose of the active drug to be administered can be easily determined by those skilled in the art. For example, an effective dose of the active drug is determined taking into consideration various factors such as kinds of disease, severity of disease, contents of active ingredients and other components contained in the composition, kinds of formulations, age, weight, health status, sex and dietary habits of patients, administration times and routes, release rates of the composition, treatment duration, and co-administered drugs.

In the present invention, an effective dose (ED) of Substance-P may be in a range of 0.1 to 100 μg/kg.

Further, an effective dose (ED) of mesenchymal stem cells, which are used in combination with Substance-P, may be in a range of 3×10⁴ to 3×10⁷ cells/kg, particularly 1×10⁶ to 1×10⁷ cells/kg.

In the method for prevention or treatment of bone marrow damage in accordance with the present invention, Substance-P may be administered in the form of a composition or medicament for prevention or treatment of bone marrow damage or in the form of an anticancer supplement.

The composition, medicament or anticancer supplement of the present invention can be administered to a subject (including human) via parenteral or topical routes, such as intravenous injection, subcutaneous injection, endothelial injection, intramuscular injection, etc. Particularly preferred is intravenous injection.

These and other objects, advantages and features of the present invention will become apparent from the detailed embodiments given below which are made in conjunction with the following Examples. The present invention may be embodied in different forms and should not be misconstrued as being limited to the embodiments set forth herein, and those skilled in the art will appreciate that various modifications, additions and substitutions are possible without departing from the scope and spirit of the invention as disclosed in the accompanying claims. Therefore, it should be understood that the embodiments disclosed herein are provided only for illustrating the present invention and should not be construed as limiting the scope and spirit of the present invention.

### EXAMPLE 1

### Effects of Substance-P on Protection and Regeneration of Bone Marrow

### (1) Administration of Substance-P to Bone Marrow Lesions

In order to ascertain protective and regenerative effects of Substance-P on bone marrow, 8-week-old C57b1/6 mice were purchased as experimental animals and divided into three groups, each consisting of 6 animals (n=6): two experimental groups (administration of a test drug before and after induction of bone marrow damage) and a control group. For this purpose, bone marrow damage was induced by radiation in animals.

For the group used to confirm bone marrow-protective effects of Substance-P, 0.1 nmol/g of Substance-P was injected into caudal vein of mice, 1 day prior to radiation. 24 hours later, animals were irradiated with a gamma irradiator at a dose of 4 Gy. 4 hours after irradiation, 0.1 nmol/g of Substance-P diluted in PBS was injected once more into caudal vein of mice. Substance-P was diluted in PBS prior to use thereof.

For the group used to confirm bone marrow-regenerative effects of Substance-P, 0.1 nmol/g of Substance-P was intravenously administered to caudal veins of mice immediately after animals were irradiated with gamma rays. 24 hours later, Substance-P was intravenously administered once more to animals.

### (2) Isolation of Bone Marrow Cells

Bone marrow cells were isolated from animals 3 and 7 days after irradiation. For this purpose, femurs were isolated from mice and defatted to obtain the femoral bones only. A medium was allowed to flow from one end of the bone to the other end thereof using a 26-gauge syringe. This procedure was repeated five times to ensure that bone marrow cells were completely extracted from the bone. The cells were pooled in a tube, and centrifuged at 1500 rpm for 5 min and washed two times with PBS. The viable cell count was performed and compared between the drug-administered groups (administration of Substance-P before and after irradiation) and the non-administered group.

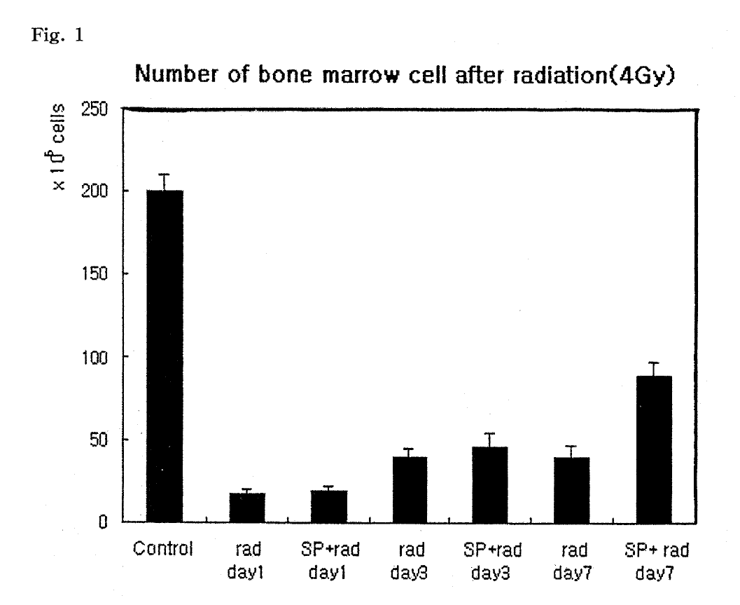

FIG. 1 is a graph showing numerical changes in viable bone marrow cells in the group to which Substance-P was administered prior to exposure of animals to radiation. Even though there was no significant difference between the irradiated and non-irradiated groups on the 3^{rd} post-irradiation day, the animal group with administration of Substance-P prior to irradiation exhibited about two-fold higher viable cell count after 7 days, as compared to the non-administered group. From these results, it can be seen that pretreatment of animals with

Substance-P can result in stimulation of MSC proliferation which then brings about the prevention of bone marrow damage.

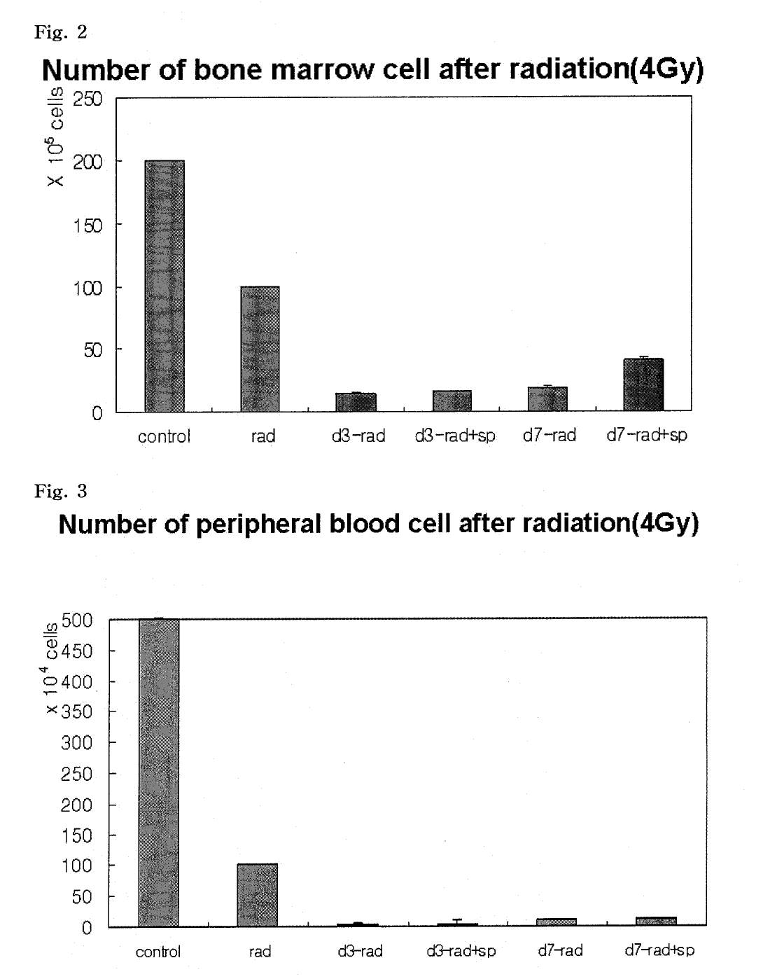

FIG. 2 is a graph showing numerical changes in viable bone marrow cells in the group to which Substance-P was administered after exposure of animals to radiation. Even though there was no significant difference between the irradiated and non-irradiated groups on the 3^{rd} post-irradiation day, the animal group with administration of Substance-P after irradiation was about 2.5-fold higher in the viable cell count after 7 days, as compared to the non-administered group. Therefore, it can be seen that administration of Substance-P after bone marrow damage can also result in stimulation of MSC proliferation which then brings about the treatment of bone marrow damage.

### (3) Isolation of Blood Cells

Blood cells were isolated from animals 3 and 7 days after irradiation treatment was complete. For this purpose, whole blood (WB) was isolated from abdominal vena cava using a 26-gauge syringe. The isolated blood was placed in a 15 mL tube and then diluted two times with addition of PBS (WelGENE Inc., Korea). For isolation of blood cells, each 2 mL of Ficoll plaque (Amersham) was placed in fresh tubes and the diluted blood was gently put on the Ficoll layer. Care was taken to ensure that the Ficoll layer was not mixed with the blood layer. After the blood layer was completely placed, centrifugation was carried out at 2200 rpm for 25 min. Once centrifugation was complete, separation of layers took place with formation of a total of four layers. These layers correspond to a plasma layer, a blood cell layer with exclusion of erythrocytes, a Ficoll layer, and an erythrocyte layer in the order of from the uppermost layer to the lowermost layer. Only the blood cell layer was separated from among them and placed in a tube to which PBS was then added, followed by centrifugation. The viable cell count was performed and compared between the drug-administered groups (administration of Substance-P before and after irradiation) and the non-administered group.

FIG. 3 shows that a rapid irreversible decrease of viable blood cells after irradiation takes place with no recovery of a normal blood cell level even after one week.

### (4) CFU Assay

When bone marrow cells and blood cells were prepared, the cells were inoculated onto 35 mm plates at a density of 3×10³ bone marrow cells/plate and 5×10⁶ blood cells/plate, respectively and cultured in hematopoietic stem cell-colony forming unit media (HSC-CFU media, available from Miltenyi). The cells were cultured without change of the culture medium for two weeks while a colony count was performed daily.

FIG. 4 is a microscopic image showing colony morphology after cell culture of bone marrow cells in HSC-CFU media, for a group with administration of Substance-P prior to exposure of animals to radiation and a group without administration. A left panel of FIG. 4 shows colony formation of the non-administered group. As can be confirmed from FIG. 4, a myriad of colonies were observed prior to irradiation, whereas the microscopic image taken on the 3^{rd} post-irradiation day showed a very low colony count and the colony number on the 7^{th} post-irradiation day increased due to regenerative function of the bone marrow itself. The group with administration of Substance-P prior to irradiation exhibited higher colony counts immediately after irradiation as well as on the 3^{rd} post-irradiation day, as compared to the non-administered group. Further, the Substance P-administered group exhibited higher colony counts on the 7^{th} post-irradiation day, as compared to the non-administered group.

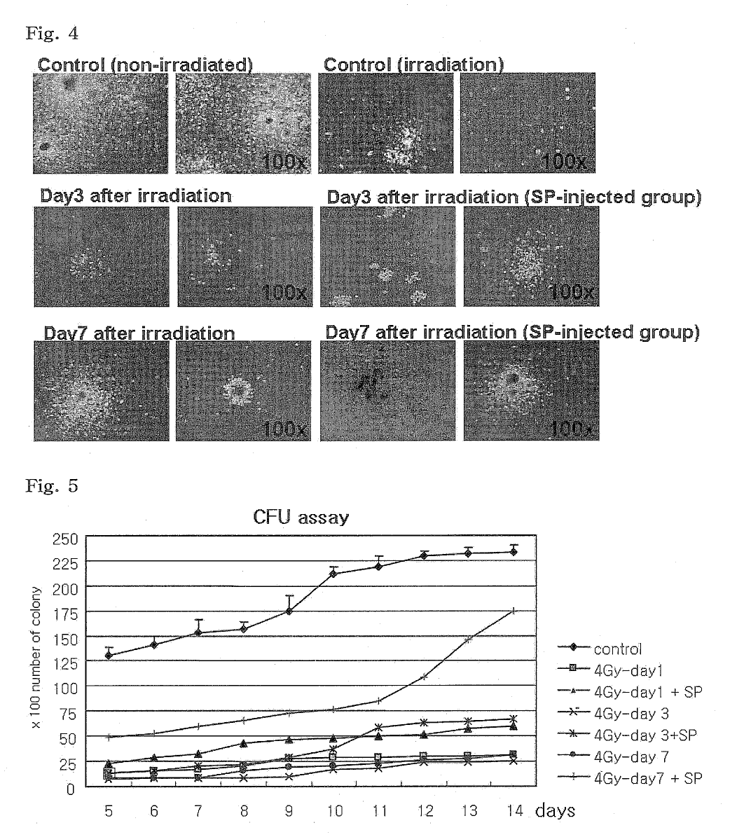

FIG. 5 is a graph showing changes in colony counts after 2-week culture of bone marrow cells in HSC-CFU media, for a group with administration of Substance-P prior to exposure of animals to radiation and a group without administration. At the early stage of cell culture, there was a slight difference in the colony count between the Substance P-administered group and the non-administered group. However, the colony count in the Substance-P pretreatment group significantly increased over time and was about 9-fold higher on the 14^{th} post-irradiation day, as compared to the non-administered group.

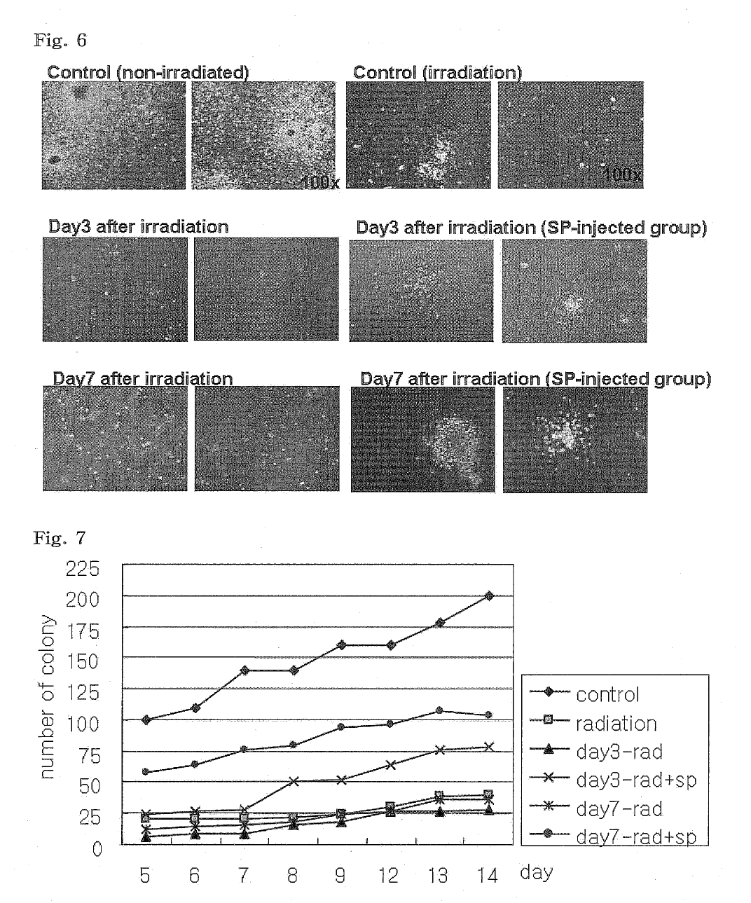

FIG. 6 is a microscopic image showing colony morphology after cell culture of bone marrow cells in HSC-CFU media, for a group with administration of Substance-P after exposure of animals to radiation and a group without administration. A left panel of FIG. 6 shows colony formation of the non-administered group. As can be confirmed from FIG. 6, the number of colonies prior to irradiation was countless, whereas the microscopic image taken on the 3^{rd} post-irradiation day exhibited no colony formation and the microscopic image on the 7^{th} post-irradiation day showed slight formation of colonies at a very insignificant number due to regenerative function of the bone marrow itself. On the other hand, the group with administration of Substance-P after irradiation exhibited no colony formation on the 3^{rd} post-irradiation day, but colony formation was observable from the 5^{th} post-irradiation day and the colony count significantly increased on the 7^{th} post-irradiation day.

FIG. 7 is a graph showing changes in colony counts after 2-week culture of bone marrow cells in HSC-CFU media, for a group with administration of Substance-P after exposure of animals to radiation and a group without administration. At the early stage of cell culture, there was a slight difference in the colony count between the Substance P-administered group and the non-administered group. However, the colony count in the

Substance P-administered group gradually increased over time and was about 3-fold higher on the 14^{th} post-irradiation day, as compared to the non-administered group.

### INDUSTRIAL APPLICABILITY

As apparent from the above description, Substance-P stimulates proliferation of mesenchymal stem cells (MSCs) within the bone marrow to thereby facilitate protection and regeneration of bone marrow cells and hematopoietic stem cells. Therefore, it is possible to achieve treatment of bone marrow damage as well as effective prevention of bone marrow damage, when the composition of the present invention comprising Substance-P as an active ingredient is therapeutically used for bone marrow damage. Further, the composition of the present invention can be used as an anticancer supplement for anticancer therapy.

1. A composition for prevention or treatment of bone marrow damage comprising Substance-P as an active ingredient.

2. The composition according to claim 1, wherein the bone marrow damage is caused by radiotherapy, chemotherapy, or trauma.

3. The composition according to claim 2, wherein the composition is administered concurrently with, before or after radiotherapy or chemotherapy.

4. The composition according to claim 1, further comprising mesenchymal stem cells (MSCs).

5. An anticancer supplement comprising Substance-P as an active ingredient.

6. (canceled)

7. (canceled)

8. (canceled)

9. (canceled)

10. (canceled)

11. A method for inhibiting, improving or treating bone marrow damage comprising administering a therapeutically effective amount of Substance-P to a mammal.

12. The method according to claim 11, wherein the bone marrow damage is caused by radiotherapy, chemotherapy or trauma.

13. The method according to claim 12, wherein Substance-P is administered concurrently with, before or after radiotherapy or chemotherapy.

14. The method according to claims 11, further comprising administering mesenchymal stem cells (MSCs).

15. A method for inhibiting, improving or treating cancer comprising administering a therapeutically effective amount of Substance-P as an anticancer supplement to a mammal.
